(19) United States
(12) Patent Application Publication
(10) Pub. No.: US 2015/0076046 A1
(43) Pub. Date: Mar. 19, 2015

(54) ANION ADSORBENT, WATER TREATMENT TANK, AND WATER TREATMENT SYSTEM

(71) Applicant: Kabushiki Kaisha Toshiba, Minato-ku (JP)

(72) Inventors: Yumiko SEKIGUCHI, Kawasaki (JP); Tomohito IDE, Inagi (JP); Hideyuki TSUJI, Yokohama (JP)

(73) Assignee: Kabushiki Kaisha Toshiba, Minato-ku (JP)

(21) Appl. No.: 14/468,546

(22) Filed: Aug. 26, 2014

(30) Foreign Application Priority Data
Sep. 18, 2013 (JP) 2013-193325

(51) Int. Cl. C07H 17/02; C02F 1/28; C08F 265/02

(52) U.S. Cl. CPC C07H 17/02; C08F 265/02; C02F 1/285; C02F 2101/12
USPC 210/96.1; 536/55.1; 536/31; 525/54.26; 525/329.9; 210/263

(57) ABSTRACT

An anion adsorbent of an embodiment includes a support and a triazine hydrochloride structure that is bound to the support.

### CROSS-REFERENCE TO RELATED APPLICATION

This application is based upon and claims the benefit of priority from Japanese Patent Applications No. 2013-193325, filed on Sep. 18, 2013; the entire contents of which are incorporated herein by reference.

### FIELD

Embodiments described herein relate to an anion adsorbent, a water treatment tank, and a water treatment system.

In recent years, due to the increasing interest in environmental cleanup and repeated utilization of resources, the importance of techniques for collecting harmful substances and valuable substances from water has been increased. Many of these substances are present in the forms of ions in water in many cases, and the ions are further divided into cations having positive electrical charge and anions having negative electrical charge. The techniques for collecting ions from water include ion exchange resins. Examples of anion exchange resins include weakly-basic anion exchange resins having an amino group at the terminal, and strongly-basic anion exchange resins having a quaternary ammonium group. The former ones are weakly basic and thus are easily regenerated, but are not capable of ion exchanging of neutral salts such as potassium iodide. On the other hand, the former ones are strongly basic and thus capable of ion exchanging of neutral salts such as potassium iodide, but are difficult to be regenerated, and a regenerant in an amount of several times of a theoretical stoichiometric amount is required for practical use. The easiness of regeneration is important for repeated use of adsorbents, and thus not only adsorption capacity but also easiness of regeneration is an important performance in ion adsorbents. In view of easiness of regeneration, weakly basic functional groups are advantageous, and functional groups having lower basicity than that of an amino group include a pyridinium group. Trimethylamine and pyridine, which are used in ion exchange units of weakly basic ion exchange resins, have base dissociation constants (pKb) of 3.2 and 8.8, respectively. A lower value of pKb shows stronger basicity. Therefore, it is understood that pyridine has significantly lower basicity than that of trimethylamine.

The pyridinium group is a heterocyclic aromatic compound in which one of the carbon atoms of a benzene ring has been replaced with a nitrogen atom. As an anion collecting agent having a heterocyclic aromatic compound, magnetic nanoparticles having a triazole group is known. However, this anion collecting agent further has an anion accepting site on the triazole group, and the anion collection ability thereof is such that the amount of chloride ion that can be collected within 1 hour per 1 g of the adsorbent is very small even under an environment in which the initial concentration of chloride ions is a relatively high concentration of 1000 mg/L.

### BRIEF DESCRIPTION OF THE DRAWINGS

FIG. 1 is a conceptual drawing showing a water treatment system using an anion adsorbent of an embodiment;

FIG. 2 is a conceptual drawing showing a water treatment tank connected to piping;

FIG. 3 is an infrared spectrum in Examples;

FIG. 4 is an infrared spectrum in Examples;

FIG. 5 is a graph showing the amount of the chloride ion eluted from the iodine adsorbent and the adsorbability for iodide ion in an embodiment;

FIG. 6 is an infrared spectrum in Examples; and

FIG. 7 is an infrared spectrum in Examples.

### DETAILED DESCRIPTION

An anion adsorbent of an embodiment includes a support and a triazine hydrochloride structure that is bound to the support.

A water treatment tank of an embodiment includes an anion adsorbent. The anion adsorbent includes a support and a triazine hydrochloride structure that is bound to the support.

A water treatment system of an embodiment includes an adsorbent unit having an anion adsorbent, a supplying unit supplying target medium water including anion for the anion adsorbent of the adsorbent unit, a discharging unit discharging the target medium water from the adsorbent unit, a measuring unit measuring concentration of an anion in the target medium water provided in the supplying unit side and/or the discharging unit side, and a controller controlling flow of the target medium water from the supplying unit to the adsorbent unit when a value calculated or obtained from a measured value in the measuring unit reaches set value. The anion adsorbent includes a support and a triazine hydrochloride structure that is bound to the support.

(Anion Adsorbent)

The anion adsorbent of an embodiment has a support, and a triazine hydrochloride structure that is bound to the support. The triazine hydrochloride structure of an embodiment is shown in the following chemical formula 1. The anion to be adsorbed is adsorbed by, for example, being exchanged for chloride ion.

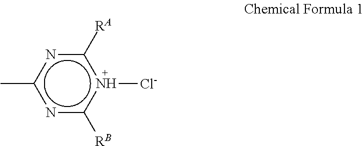

R^{A} and R^{B} are selected from H, R^{C}, OR^{C}, OM and NR^{D}R^{E}. R^{C} is an alkyl group having a carbon number of 3 or less or β-cyclodextrin. M is selected from Na and K. R^{D} and R^{E} are selected from hydrogen and an alkyl group having a carbon number of 3 or less. R^{A} and R^{B} may be identical or different. R^{D} and R^{E} may be identical or different. It is more preferable that at least one of R^{A} and R^{B} is an alkoxy group, but an alkyl group or an amino group is also preferable. This is because all of the above-mentioned substituents are electron-donating substituents, and thus they impart a similar effect that the electron density on the nitrogen atom is increased and thus the proton acceptability is improved. When the proton acceptability is improved, a hydrochloride structure (NH⁺Cl⁻) is easily formed. The R^{A} and R^{B} in the chemical formulas 2, 3 and 4 are as defined by R^{A} and R^{B} in the chemical formula 1. Hydrogen can be used since it affects little on the nitrogen. The β-cyclodextrin also includes derivatives thereof.

Furthermore, the adsorbent of an embodiment may also have a triazine structure instead of the triazine hydrochloride structure. The chemical formula 2 of the triazine structure is shown below. It is considered that the nitrogens of the triazine structure adsorb the anions in mineral acids such as HCl and H₂SO₄ as shown in, for example, the chemical formula 3.

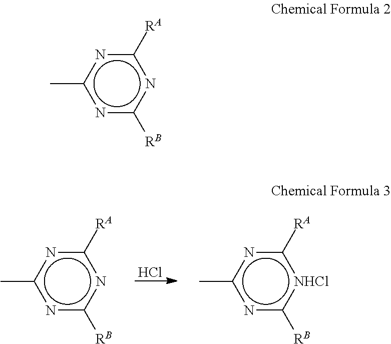

The support is not especially limited as long as it supports the triazine hydrochloride structure. As the support, organic supports and inorganic supports can be used. The support and the triazine hydrochloride structure are connected by an organic backbone having a carbon chain. In the case when the adsorbent is synthesized by reacting a compound having a triazine hydrochloride structure having a halogen as a substituent and the support, it is preferable that the organic backbone has carbon, hydrogen and oxygen, as well as an amine that reacts with the halogen. The organic backbone may also contain silane derived from, for example, a silane coupling agent, and the like.

As the organic support, polymer compounds such as acrylic resins and chitosan can be used. Since the acrylic resins have high mechanical strength and have ester bond moieties, amino groups can be introduced therein by a transesterification reaction with various amines such as ethylenediamine and diethylenetriamine.

Examples of the inorganic supports can include silica (SiO₂), titania (TiO₂), alumina (Al₂O₃) and zirconia (ZrO₂), and alkoxides, halides and the like that form cobalt trioxide (CoO₃), cobalt oxide (CoO), tungsten oxide (WO₃), molybdenum oxide (MoO₃), indium tin oxide (ITO), indium oxide (In₂O₃), lead oxide (PbO₂), PZT, niobium oxide (Nb₂O₅), thorium oxide (ThO₂), tantalum oxide (Ta₂O₅), calcium titanate (CaTiO₃), lanthanum cobaltate (LaCoO₃), rhenium trioxide (ReO₃), chromium oxide (Cr₂O₃), iron oxide (Fe₂O₃), lanthanum chromate (LaCrO₃), barium titanate (BaTiO₃) and the like.

Amino groups are imparted to these inorganic supports by generally a reaction of a silane coupling agent having an amino group and the inorganic support. Therefore, among the above-mentioned inorganic supports, silica, titania, alumina and zirconia are preferable since the ratio of the hydroxyl groups on the surface is high, and thus a higher surface modification rate can be obtained by a silane coupling reaction. As silane coupling agents having an amino group, hydrochlorides of N-2-(aminoethyl)-3-aminopropylmethyldimethoxysilane, N-2-(aminoethyl)-3-aminopropyltrimethoxysilane, N-2-(aminoethyl)-3-aminopropyltriethoxysilane, 3-aminopropyltrimethoxysilane, 3-aminopropyltriethoxysilane, 3-triethoxysilyl-N-(1,3-dimethyl-butyridene)propylamine, N-phenyl-3-aminopropyltrimethoxysilane, N-(vinylbenzyl)-2-aminoethyl-3-aminopropyltrimethoxysilane and the like are practical, and it is considered that similar effects can be obtained by using analogs in which the length of the alkoxy group or alkyl chain is different in these silane coupling agents. The reaction solvent may be any reaction solvent that can dissolve the above-mentioned silane coupling agents having an amino group.

The triazine hydrochloride structure can be introduced by a reaction between the above-mentioned support having an amino group and a chlorotriazine having a structure of the chemical formula 4. The introduction of the triazine hydrochloride structure into the support by the reaction between the support having an amino group and the chlorotriazine is exemplified, but the introduction is not limited to this.

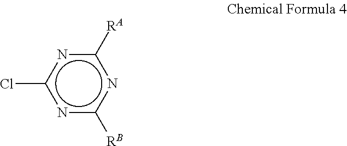

The size of the anion adsorbent in this embodiment is preferably an average primary particle size of 100 μm or more and 5 mm or less. If the average primary particle size of the anion adsorbent is set to 100 μm or more and 5 mm or less, for example, a high filling rate of the anion adsorbent in a column, a cartridge or a tank and easiness of water passing can be achieved at the same time when anion adsorption is conducted. If the average primary particle size is lower than 100 μm, the filling rate of the anion adsorbent in a column or the like becomes too high and the rate of airspaces decreases, and thus it becomes difficult to conduct water passing. On the other hand, if the average primary particle size exceeds 5 mm, the filling rate of the anion adsorbent in a column or the like becomes too low and the airspaces increase; therefore, it becomes easy to conduct water passing, whereas the contact surface area between the anion adsorbent with the anion-containing discharged water decreases, and thus the adsorption ratio of the anion by the anion adsorbent is decreased. The support has an average primary particle size of preferably 100 μm or more and 2 mm or less, more preferably 300 μm or more and 1 mm or less.

The average primary particle size can be measured by a sieving method. Specifically, the average primary particle size can be measured according to JIS Z 8901: 2006 “Powder Body for Test and Particles for Test” by using plural sieves having different openings.

The anion adsorbent of an embodiment can adjust the size of the adsorbent itself by only changing the size of the support, and thus it is understood that it is only necessary to preset the size of the support to a predetermined size so as to obtain an adsorbent that is easily handled. Namely, an anion adsorbent that is easily handled can be obtained without conducting an operation such as granulation. Furthermore, since it is not necessary to conduct granulation or the like, the production steps that are necessary to obtain an anion adsorbent that is easily handled can be simplified, and thus the costs can be decreased.

(Anion Adsorption System and Method for Using Anion Adsorbent)

Next, an adsorption system and a method for using the adsorption system with the above-mentioned anion adsorbent will be explained. The anion treatment system includes an adsorbent unit having an anion adsorbent, a supplying unit configured to supply a target medium water containing an anion for the anion adsorbent of the adsorbent unit, a discharging unit configured to discharge the target medium water from the adsorbent unit, a measuring unit configured to measure the concentration of the anion in the target medium water, which is provided (disposed) in at least one of the supplying unit side and/or the discharging unit side of the adsorbent unit, and a controller configured to control flow of the target medium water from the supplying unit to the adsorbent unit when a value calculated or obtained from a measured value in the measuring unit reaches a set value.

The anion adsorbing method of an embodiment is such that anion-containing water is brought into contact with a tank containing the anion adsorbent.

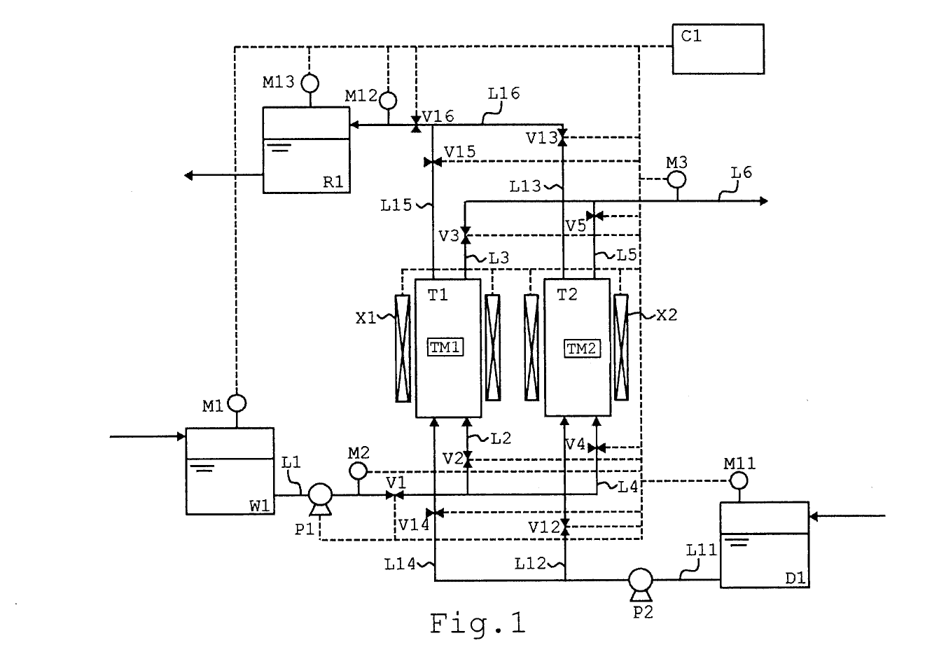

FIG. 1 is a conceptual drawing showing the schematic constitution and the treatment system of the apparatus used for anion adsorption in this embodiment.

As shown in FIG. 1, in this apparatus, water treatment tanks (adsorbent units) T1 and T2 in which the above-mentioned anion adsorbent is filled are disposed in parallel, and contact efficiency promoters X1 and X2 are disposed outside of the water treatment tank T1 and T2. The contact efficiency promoters X1 and X2 can be mechanical stirrers or non-contacting magnetic stirrers, but are not essential constitutional elements and thus can be omitted.

Furthermore, a discharged water storage tank W1 in which anion-containing discharged water (a target medium water) is stored is connected to the water treatment tanks T1 and T2 through discharged water-supplying lines (supplying units) L1, L2 and L4, and the water treatment tanks T1 and T2 are connected to outside through discharged water-discharging lines (discharging units) L3, L5 and L6.

Valves (controllers) V1, V2 and V4 are respectively disposed on the supplying lines L1, L2 and L4, and valves (controllers) V3 and V5 are respectively disposed on the discharging lines L3 and L5. Furthermore, a pump (controller) P1 is disposed on the supplying line L1. Furthermore, concentration measuring units (measuring units) M1, M2 and M3 are disposed on the discharged water storage tank W1, supplying line L1 and discharging line L6, respectively.

Furthermore, the control of the above-mentioned valves and pump and the monitoring of measured values in the measurement apparatuses are collectively subjected to centralized control by a controller C1.

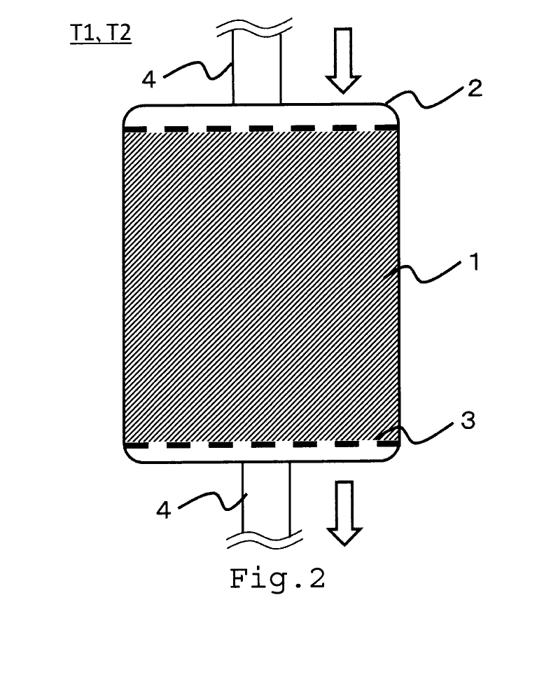

FIG. 2 shows a conceptual cross-sectional drawing of the water treatment tanks T1 and T2 in which the anion adsorbent is filled, which are connected to the piping 4 (L2-L4). The arrows in the drawing indicate the direction of the flow of the treated water. The water treatment tanks T1 and T2 are each constituted by an anion adsorbent 1, a tank 2 configured to house the anion adsorbent, and a partition plate 3 configured to prevent the anion adsorbent from leaking out of the tank 2. The water treatment tanks T1 and T2 may have a cartridge-type form in which the tank 2 itself can be replaced, or may have forms in which the anion adsorbent in the tank 2 can be replaced. In the case when there is a substance to be adsorbed and collected other than the anion, another adsorbent can be housed in the tank 2.

Next, the operation of adsorbing an anion by using the apparatus shown in FIG. 1 will be explained.

Firstly, for the water treatment tanks T1 and T2, discharged water is supplied to the water treatment tanks T1 and T2 from the tank W1 by the pump P1 through the discharged water-supplying lines L1, L2 and L4. At this time, the anion in the discharged water is adsorbed by the water treatment tanks T1 and T2, and the discharged water after the adsorption is discharged outside through the discharged water-discharging lines L3 and L5.

At this time, where necessary, the contact efficiency promoters X1 and X2 are driven to increase the contact surface area between the anion adsorbent filled in the water treatment tanks T1 and T2 and the discharged water, whereby the anion adsorption efficiency by the water treatment tanks T1 and T2 can be improved.

Here, the adsorption states of the water treatment tanks T1 and T2 are observed by a concentration measuring unit M2 disposed on the supply side and a concentration measuring unit M3 disposed on the discharge side of the water treatment tanks T1 and T2. In the case when the adsorption is smoothly conducted, the concentration of the anion measured by the concentration measuring unit M3 shows a lower value than the concentration of the anion measured by the concentration measuring unit M2. However, as the adsorption of the anion in the water treatment tanks T1 and T2 gradually progresses, the difference in the concentrations of the anions in the concentration measuring units M2 and M3 disposed on the supply unit and discharge unit is decreased.

Therefore, in the case when it is judged that the concentration measuring unit M3 has reached the predetermined value preset in advance and the adsorbability for the anion by the water treatment tanks T1 and T2 has reached saturation, the controller C1 once stops the pump P1, closes the valves V2, V3 and V4, and stops the supply of the discharged water to the water treatment tanks T1 and T2, based on the information from the concentration measuring units M2 and M3.

Although not shown in FIG. 1, in the case when the pH of the discharged water varies, or in the case when the pH is strongly acidic or strongly alkaline and thus is out of the pH range suitable for the adsorbent according to this embodiment, it is also possible to measure the pH of the discharged water by the concentration measuring unit M1 or/and M2, and adjust the pH of the discharged water through the controller C1.

After the attainment of saturation of the water treatment tanks T1 and T2, the water treatment tanks are suitably replaced with new water treatment tanks in which an anion adsorbent is filled, and the water treatment tanks T1 and T2 in which the anion adsorption has reached saturation are suitably subjected to a necessary post treatment. For example, in the case when the water treatment tanks T1 and T2 contain radioactive iodine, for example, the water treatment tanks T1 and T2 are pulverized, subjected to cement solidification, and stored as a radioactive waste material in an underground facility or the like.

Although a system and operations for adsorbing an anion in discharged water using water treatment tanks are explained in the above-mentioned example, it is also possible to remove an anion in an exhaust gas by aerating the above-mentioned column with an anion-containing exhaust gas.

Hereinafter the adsorbent will be specifically explained by Examples.

### Example 1

### Synthesis of Acrylic Particles Graft-Modified with Amino Groups

0.08 g of a polyvinyl alcohol (hereinafter referred to as PVA), 19.6 g of sodium chloride and 500 mL of ion exchanged water were added to a recovery flask (1 L capacity) equipped with a magnetic stirrer bar, and stirred at room temperature (25° C.) for 40 minutes to give a colorless solution. Then, a Dimroth condenser was attached to the flask, and the inside of the system was deaerated and substituted with nitrogen.

Subsequently, 17.8 mL of methacrylic acid, 6 mL of divinylbenzene, 34 mL of chlorobenzene and 0.2 g of azobisisobutyronitrile (hereinafter AIBN) were put into a beaker (200 mL capacity) and mixed. The obtained mixture was added to the above-mentioned solution by decantation, and the mixture was stirred in a nitrogen atmosphere under heating at 80° C. for 6 hours in an oil bath to give a colorless suspension. The flask was removed from the oil bath, and the suspension was exposed to air to thereby inactivate the AIBN.

Subsequently, the suspension was left still in a draft to thereby precipitate resin microparticles, and the supernatant was removed by decantation. Ion exchanged water having a similar volume accuracy to that of the remaining resin microparticles was put into the resin microparticles, the resin microparticles was washed by lightly shaking the flask with the hand, and the supernatant was removed by decantation. This washing operation was repeatedly conducted three times. The obtained resin microparticles were subjected to aspiration filtration by using a Kiriyama funnel, and washed with ion exchanged water and acetone in this order. Finally, the solvent was completely distilled off under a reduced pressure to give an acrylic resin support.

Subsequently, 5 g of the acrylic resin support was put into a two-necked recovery flask (100 mL capacity) equipped with a magnetic stirrer bar and a Dimroth condenser, and deaeration and substitution with nitrogen were repeatedly conducted three times. Furthermore, 20 mL of ethylenediamine was added under a nitrogen atmosphere, and stirring was conducted under heating at 120° C. for 9 hours. The temperature was returned to room temperature, and the product was filtered by aspiration using a Kiriyama funnel and washed with ion exchanged water and acetone in this order. Subsequently, the solvent was completely distilled off under a reduced pressure to give a pale yellow acrylic resin support graft-modified with amino groups.

(Synthesis of Anion Adsorbent)

0.150 g of the acrylic polymer graft-modified with amino groups, 1.50 g of a Sodium chloride-containing powder of monochlorotriazino-β-cyclodextrin and 15 ml of pure water were put into a 20-mL screw vial, and stirred by a horizontal mix rotor (number of rotation 60 rpm) at room temperature for 7 hours. Subsequently, the obtained microparticles were filtered and washed with pure water, and the solvent was distilled off over 7 hours to give an anion adsorbent as reddish brown microparticles (yield 0.165 g).

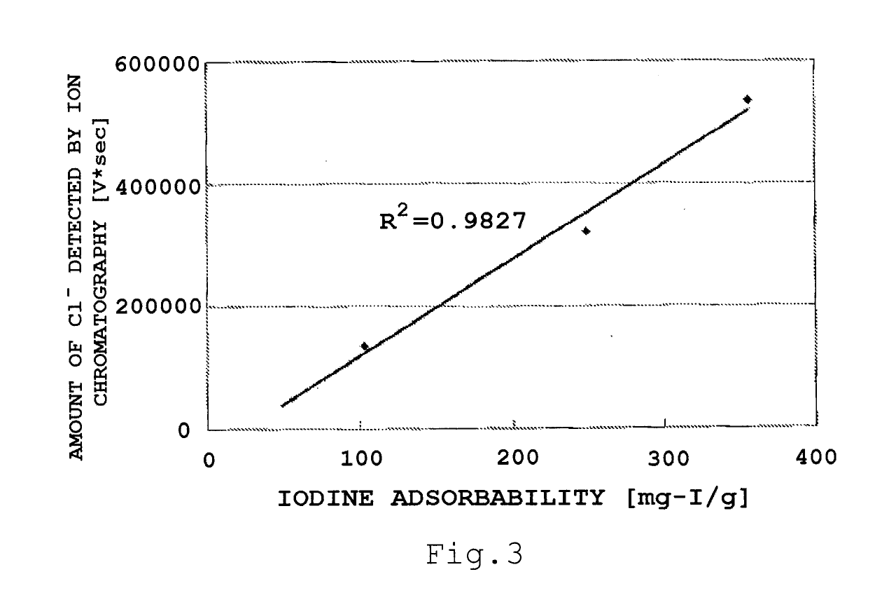

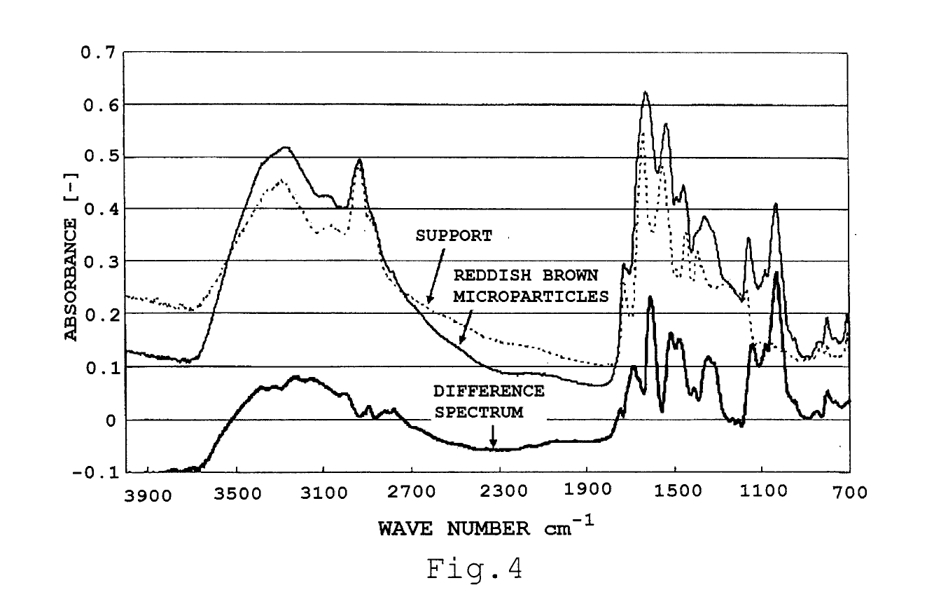

The structure of the obtained reddish brown microparticles was identified by using infrared spectra. FIG. 3 collectively shows the spectrum of the obtained reddish brown microparticles, the spectrum of the support before the reaction, and the difference spectrum obtained by subtracting the spectrum of the support from the spectrum of the reddish brown microparticles. Furthermore, FIG. 4 collectively shows the difference spectrum of FIG. 3 and the spectrum of the monochlorotriazino-β-cyclodextrin.

It was clarified by FIG. 4 that the difference spectrum of the obtained reddish brown microparticles and the support conforms well with the spectrum of the monochlorotriazino-β-cyclodextrin. Furthermore, a peak that is considered to be derived from the triazine ring was clearly confirmed at 800 cm⁻¹, and a peak that is characteristic to polysaccharides and considered to be derived from C—O stretch vibration was clearly confirmed at 1100 cm⁻¹. The above-mentioned result indicated that the obtained reddish brown microparticles had a cyclodextrin and a triazine ring.

(Anion Adsorbing Test)

20 mg of the synthesized anion adsorbent was put into a 30-mL screw vial, 20 mL of a 650 ppm aqueous solution of potassium iodide (KI) was added thereto, and the mixture was stirred at room temperature for 2 hours by using a horizontal mix rotor (number of rotation: 60 rpm). The adsorbent was filtered by a membrane filter made of cellulose, the and the anion (I⁻) concentration in the obtained colorless solution was calculated by using ion chromatography. As an ion chromatography apparatus, an Alliance HPLC system manufactured by Japan Waters was used, and the measurement was conducted under the following conditions. The result of the adsorption test was shown in Table 1.

Column Shodex IC SI-90 4E

Eluant 1.8 mM Na2CO3+1.7 mM NaHCO3 aq.

Flux 1.2 mL/min

Detector Shodex CD Suppressor module

Column temperature 30° C.

As an index of the adsorbability for the iodide ion, an iodide ion adsorption amount per unit weight (hereinafter referred to as mg−I/g) was used. The mg−I/g is the amount of the iodide ion that can be collected by 1 g of the used anion adsorbent, and is derived by the following formula.

mg−I/g=([I⁻]₀−[I⁻])/C

[I⁻]₀: the initial concentration of I⁻ (mg/L)
[I⁻]: the final concentration of I⁻ (mg/L)
C: the concentration of the anion adsorbent (g/L)

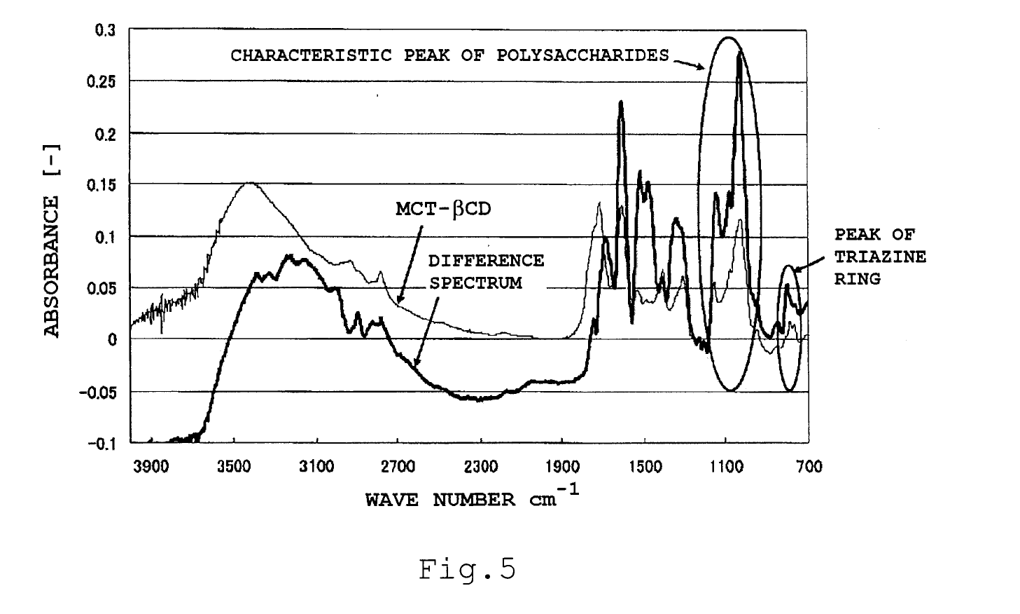

FIG. 5 is a graph showing the relationship between the amount of the chloride ion eluted from the anion adsorbent and the adsorbability for the iodide ion by the anion adsorbent in the case when the anion adsorbent of Example 1 is immersed in a solution containing iodide ion. The method for measuring the adsorption amount is similar to the above-mentioned anion adsorption test. As is apparent from FIG. 5, as the amount of the chloride ion eluted into the ion exchanged water increased, the adsorbability for the iodide ion in the solution increased. Also from this fact, it is presumed that the iodide ion in the test solution is adsorbed by the ion exchanging with the chloride ion of the anion adsorbent.

### Example 2

A reaction using chitosan (manufactured by Wako Pure Chemical Industries, Ltd.) as a support instead of the acrylic polymer graft-modified with amino groups was conducted to give an anion adsorbent as a colorless solid.

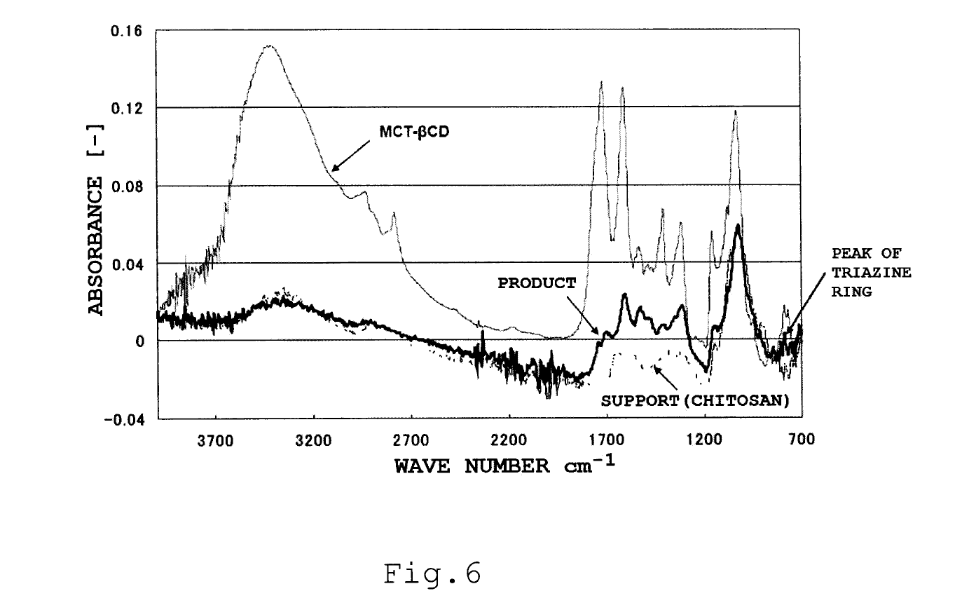

The obtained compound was identified by using infrared spectra. FIG. 6 collectively shows the spectrum of the product in the case when chitosan was used as a support, and the spectra of the support (chitosan) before the reaction and monochlorotriazino-β-cyclodextrin (MCT-β-CD). Since chitosan is also a polysaccharide, no difference was able to be confirmed before and after the reaction in the peak around 1100 cm⁻¹ that is characteristic to the polysaccharide, whereas a peak around 800 cm⁻¹ that is considered to be derived from the triazine ring was observed in the spectrum of the product. Furthermore, also from 1200 cm⁻¹ to 1700 cm⁻¹, a peak was confirmed at a similar position to that for monochlorotriazino-β-cyclodextrin.

It was shown by the above-mentioned result that the product had β-cyclodextrin and a triazine ring also in the case when chitosan was used as a support.

Furthermore, an anion adsorption test was conducted in a similar manner to Example 1. The result of the adsorption test is shown in Table 1.

### Comparative Example 1

Monochlorotriazino-β-cyclodextrin was reacted with porous cellulose particles (Viscopearl manufactured by Rengo Co., Ltd.) in a similar manner to Example 1 to give colorless particles. Furthermore, an anion adsorption test was conducted in a similar manner to Example 1. The result of the adsorption test is shown in Table 1.

In the case when porous cellulose particles were used as a support, the IR spectrum of the product was almost identical with the spectrum of the support before the reaction. This result suggests that, in the case when the porous cellulose particles are used as a support, the reaction with the monochlorotriazino-β-cyclodextrin is difficult to progress.

### Comparative Example 2

An anion adsorption test was conducted in a similar manner to Example 1 by using the monochlorotriazino-β-cyclodextrin used in Example 1 in its original form without being bound to a support. The result of the adsorption test is shown in Table 1.

### Comparative Example 3

An anion adsorption test was conducted on β-cyclodextrin in a similar manner to Example 1. The result of the adsorption test is shown in Table 1.

### Comparative Example 4

An anion adsorption test was conducted in a similar manner to Example 1 by using the acrylic polymer graft-modified with amino groups used in Example 1 in its original form. The result of the adsorption test is shown in Table 1.

### Comparative Example 5

An anion adsorption test was conducted in a similar manner to Example 1 by using the chitosan in Example 2 in its original form without reacting with monochlorotriazino-β-cyclodextrin. The result of the adsorption test is shown in Table 1.

### Comparative Example 6

An anion adsorption test was conducted in a similar manner to Example 1 by using the porous cellulose used in Comparative Example 1 in its original form. The result of the adsorption test is shown in Table 1.

### Example 3

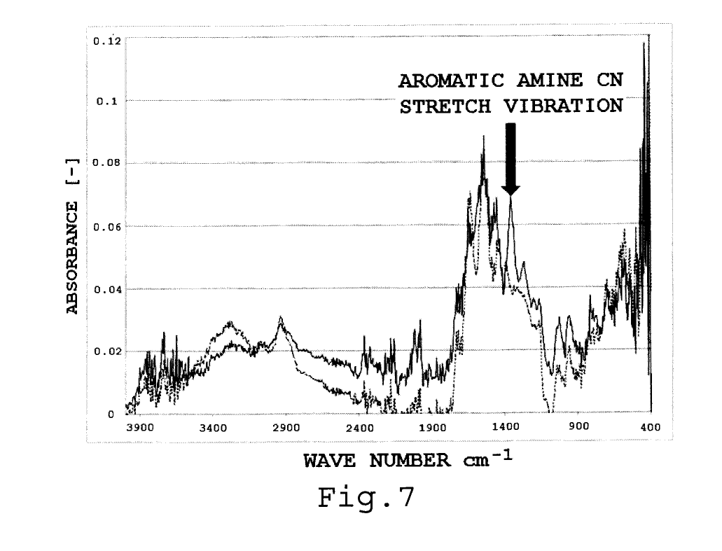

The ethylenediamine terminal-modified acrylic resin synthesized in Example 1 (0.100 g) was put into a recovery flask (50 mL) equipped with a magnetic stirrer bar, and the flask was deaerated and then substituted with nitrogen. 2-Chloro-4,6-dimethoxytriazine (0.114 g) and methanol (10 mL) were put therein, and stirring was conducted under a nitrogen atmosphere at room temperature for 27 hours. The obtained reddish brown particles were filtered and washed with methanol (20 mL). The solvent was distilled off under a reduced pressure to give the titled compound as reddish brown microparticles (yield 0.184 g). The introduction of the triazine group was confirmed by infrared absorption spectra. FIG. 7 collectively shows the infrared absorption spectra before and after the reaction with chlorotriazine. In the spectrum after the reaction (solid line), a peak was confirmed in the vicinity of 1360 cm⁻¹, and this attributed to the CN stretch vibration of the aromatic amine.

The synthesized anion adsorbent (20 mg) and an aqueous potassium iodide solution containing iodide ion at a concentration of 500 ppm (20 mL) were added to a screw vial (30 mL) and stirred at room temperature for 1 hour by using a horizontal mix rotor (number of rotation: 60 rpm). The product was filtered by a membrane filter made of cellulose, and the iodide ion concentration in the obtained colorless solution was measured by using ion chromatography in a similar manner to Example 1. The result of the adsorption test is shown in Table 1.

### Comparative Example 7

An anion adsorption test was conducted in a similar manner to Example 3 by using the acrylic polymer graft-modified with amino groups used in Example 1 in its original form. The result of the adsorption test is shown in Table 1.

TABLE 1

| | | | Adsorbed |
|---|---|---|---|
| | | Adsorptive | amount of I |
| | Support | group | [mg-I/g] |
| Example 1 | Acrylic polymer | Triazino-β- | 249 |
| | graft-modified | cyclodextrin | |
| | with amino | | |
| | groups | | |
| Example 2 | Chitosan | Triazino-β- | 103 |
| | | cyclodextrin | |
| Comparative | Cellulose | Triazino-β- | 1.34 |
| Example 1 | particles | cyclodextrin | |
| Comparative | — | Triazino-β- | 60.6 |
| Example 2 | | cyclodextrin | |
| Comparative | — | β-cyclodextrin | 11.2 |
| Example 3 | | | |
| Comparative | Acrylic polymer | — | 74 |
| Example 4 | graft-modified | | |
| | with amino | | |
| | groups | | |
| Comparative | Chitosan | — | 7.96 |
| Example 5 | | | |
| Comparative | Cellulose | — | 62 |
| Example 6 | particles | | |
| Example 3 | Acrylic polymer | Dimethoxytriazino | 168 |
| | graft-modified | | |
| | with amino | | |
| | groups | | |
| Comparative | Acrylic polymer | — | 43 |
| Example 7 | graft-modified | | |
| | with amino | | |
| | groups | | |

It was found from Table 1 that the adsorbed amount of the anion is significantly improved by introducing a triazino structure (hydrochloride). Furthermore, since the adsorbed amount of the anion is further increased by introducing cyclodextrins in the adsorptive group, it is considered that cyclodextrins also have anion adsorbability. Although iodide ion is explained as an example of an anion in Examples, it is considered that the adsorbent of the embodiment also adsorbs anions other than iodide ion, in view of the phenomenon of ion exchanging with chloride ion and the adsorption mechanism of the triazine structure.

Some of the elements in the specification are represented by elemental symbols.

While certain embodiments have been described, these embodiments have been presented by way of Example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An anion adsorbent comprising:
a support; and
a triazine hydrochloride structure that is bound to the support.

2. The anion adsorbent according to claim 1, wherein the triazine hydrochloride structure is a structure of the chemical formula 1,
wherein R^{A} and R^{B} in the chemical formula are selected from H, R^{C}, OR^{C}, OM and NR^{D}R^{E},
wherein R^{C} is an alkyl group having a carbon number of 3 or less,
M is selected from Na and K, and
R^{D} and R^{E} are selected from hydrogen and an alkyl group having a carbon number of 3 or less.

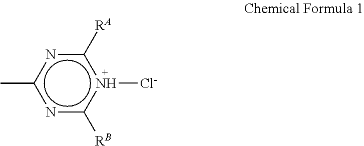

3. A water treatment tank comprising:
a an anion adsorbent,
wherein the anion adsorbent comprising a support and a triazine hydrochloride structure that is bonded to the support.

4. The tank according to claim 3, wherein the triazine hydrochloride structure is a structure of the chemical formula 1,
wherein R^{A} and R^{B} in the chemical formula are selected from H, R^{C}, OR^{C}, OM and NR^{D}R^{E},
wherein R^{C} is an alkyl group having a carbon number of 3 or less,
M is selected from Na and K, and
R^{D} and R^{E} are selected from hydrogen and an alkyl group having a carbon number of 3 or less.

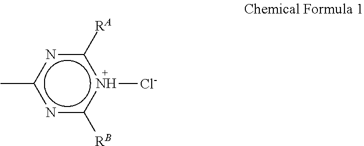

5. A water treatment system comprising:
an adsorbent unit having an anion adsorbent;
a supplying unit supplying target medium water including anion for the anion adsorbent of the adsorbent unit;
a discharging unit discharging the target medium water from the adsorbent unit;
a measuring unit measuring concentration of an anion in the target medium water provided in the supplying unit side and/or the discharging unit side; and
a controller controlling flow of the target medium water from the supplying unit to the adsorbent unit when a value calculated or obtained from a measured value in the measuring unit reaches set value,
wherein the anion adsorbent comprising a support and a triazine hydrochloride structure that is bound to the support.

6. The system according to claim 5, wherein the triazine hydrochloride structure is a structure of the chemical formula 1,
wherein R^{A} and R^{B} in the chemical formula are selected from H, R^{C}, OR^{C}, OM and NR^{D}R^{E},
wherein R^{C} is an alkyl group having a carbon number of 3 or less,
M is selected from Na and K, and
R^{D} and R^{E} are selected from hydrogen and an alkyl group having a carbon number of 3 or less. [chemical structure]
